(19) United States
(12) Patent Application Publication
(10) Pub. No.: US 2008/0086097 A1
(43) Pub. Date: Apr. 10, 2008

(54) VASCULAR ACCESS DEVICE FLUID FLOW DIRECTION

(75) Inventors: Wayne K. Rasmussen, Riverdale, UT (US); Weston F. Harding, Lehi, UT (US); Bryan G. Davis, Sandy, UT (US); Austin Jason McKinnon, Herriman, UT (US); Lantao Guo, Draper, UT (US); Kelly D. Christensen, Centerville, UT (US)

Correspondence Address: David W. Highet;Becton, Dickinson and Company, (Metcalf Intellectual Property Law, LLC), 1 Becton Drive, MC 110, Franklin Lakes, NJ, 07417-1880, US

(73) Assignee: BECTON, DICKINSON AND COMPANY, Franklin Lakes, NJ (US)

(21) Appl. No.: 11/864,289

(22) Filed: Sep. 28, 2007

Related U.S. Application Data
(60) Provisional application No. 60/828,354, filed on Oct. 5, 2006.

(51) Int. Cl. A61M 5/14

(52) U.S. Cl. 604/266

(57) ABSTRACT

A medical device may include an extravascular system for communication of fluid with a vascular system, a fluid path within the extravascular system, and a fluid flow director in communication with the fluid path. The director encourages movement of stagnant fluid within the fluid path of the extravascular system. A method may include providing an extravascular system having a fluid path and encouraging the movement of stagnant fluid within the fluid path of the extravascular system.

### RELATED APPLICATIONS

This application claims the benefit of U.S. Provisional Application No. 60/828,354, filed Oct. 5, 2006, entitled VASCULAR ACCESS DEVICE FLUID FLOW DIRECTION, which is incorporated herein by reference.

### BACKGROUND OF THE INVENTION

The present disclosure relates to fluid flow direction in extravascular systems used to provide infusion or other therapy to patients. Infusion therapy is one of the most common health care procedures. Hospitalized and home care patients receive fluids, pharmaceuticals, and blood products via a vascular access device inserted into the vascular system. Infusion therapy may be used to treat an infection, provide anesthesia or analgesia, provide nutritional support, treat cancerous growths, maintain blood pressure and heart rhythm, or many other clinically significant uses.

Infusion therapy is facilitated by vascular access devices located outside the vascular system of a patient. An extravascular system includes at least one vascular access device and/or other medical device that may access a patient's peripheral or central vasculature, either directly or indirectly. Vascular access devices include closed access devices, such as the BD Q-SYTE™ closed Luer access device of Becton, Dickinson and Company; syringes; split access devices; catheters; and intravenous (IV) fluid chambers. An extravascular system may access a patient's vascular system for a short term (days), a moderate term (weeks), or a long term (months to years), and may be used for continuous infusion therapy or for intermittent therapy.

Complications associated with infusion therapy include significant morbidity and even mortality. Such complications may be caused by regions of stagnant fluid flow within the vascular access device or nearby areas of the extravascular system. These are regions in which the flow of fluid is limited or non-existent due to the conformation of the extravascular system or the fluid dynamics within that area of the extravascular system. Air bubbles or infused medications may become trapped within these regions of stagnant flow as a result of the limited or non-existent fluid flow. When a different medication is infused into the extravascular system, or the extravascular system is exposed to physical trauma, the extravascular system's fluid flow may become altered, releasing trapped air bubbles or residual medications back into the active fluid path of the extravascular system. This release of air bubbles and residual medication into the active fluid path extravascular system may result in significant complications.

Released air bubbles may block fluid flow through the extravascular system and prevent its proper functioning. More seriously, released air bubbles may enter the vascular system of the patient and block blood flow, causing tissue damage and even stroke. In addition, residual medications may interact with presently infused medications to cause precipitates within the extravascular system and prevent its proper functioning. Furthermore, residual medications may enter the vascular system of the patient and cause unintended and/or undesired effects.

Therefore, a need exists for systems and methods that eliminate, prevent, or limit regions of stagnant flow within vascular access devices and extravascular systems.

### BRIEF SUMMARY OF THE INVENTION

The present invention has been developed in response to problems and needs in the art that have not yet been fully resolved by currently available extravascular systems, devices, and methods. Thus, these developed systems, devices, and methods provide an extravascular system that may be connected to a patient's vascular system and will eliminate, prevent, or limit regions of stagnant flow within the vascular access device or the extravascular system by strategically directing fluid flow.

A medical device may include an extravascular system for communication of fluid with a vascular system, a fluid path within the extravascular system, and a fluid flow director in communication with the fluid path. The director may encourage the movement of stagnant fluid within the fluid path of the extravascular system. The fluid flow director may include a variety of embodiments capable of encouraging the movement of stagnant fluid within the fluid path of the extravascular system.

The fluid flow director may include an arch, a rotatable arch with a lip at the tip of the arch, an arch and a flow channel, an arch that defines a radial fluid path, an arm, an offset input, a valve, a septum having a duck bill oriented parallel to the fluid path, a venturi, a pointed floor having an offset outlet hole, a ramp and a helical floor having an offset outlet hole, an offset outlet hole and a wall surrounding a portion of the offset outlet hole, a turbine, an inlet torous and a goblet-shaped insert, an outlet torous, a cup-shaped barrier having an outlet at an edge of the cup, a deflectable membrane and a Luer tip receiver, a hydrophilic material, a hydrophobic material, and/or a soluble material.

A method may include providing an extravascular system having a fluid path, and encouraging the movement of stagnant fluid within the fluid path of the extravascular system. A medical device may include a means for accessing the vascular system of a patient and a means for encouraging the movement of stagnant fluid. The means for encouraging the movement of stagnant fluid may at least partially reside within the means for accessing the vascular system of the patient.

These and other features and advantages of the present invention may be incorporated into certain embodiments of the invention and will become more fully apparent from the following description and appended claims, or may be learned by the practice of the invention as set forth hereinafter. The present invention does not require that all the advantageous features and all the advantages described herein be incorporated into every embodiment of the invention.

### BRIEF DESCRIPTION OF THE SEVERAL VIEWS OF THE DRAWINGS

In order that the manner in which the above-recited and other features and advantages of the invention are obtained will be readily understood, a more particular description of the invention briefly described above will be rendered by reference to specific embodiments thereof which are illustrated in the appended drawings. These drawings depict only typical embodiments of the invention and are not therefore to be considered to limit the scope of the invention.

FIG. 1 is a cross section view of a fluid flow director including an arch.

FIG. 2 is a perspective cross section view of a fluid flow director including a rotatable arch.

FIG. 3 is another perspective cross section view of the fluid flow director including the rotatable arch of FIG. 2.

FIG. 4 is a cross section view of the fluid flow director including the rotatable arch of FIGS. 2 and 3.

FIG. 5 is a cross section view of a fluid flow director including an arch and a flow channel.

FIG. 5a is a cross section end view of the device of FIG. 5.

FIG. 6 is a cross section view of a fluid flow director including an arch that defines a radial fluid path.

FIG. 7 is a perspective cross section view of a fluid flow director including an arm.

FIG. 8 is a cross section view of a fluid flow director including an offset input.

FIG. 9 is a cross section view of the fluid flow director including the offset input of FIG. 8 taken along lines A-A.

FIG. 10 is a cross section view of a fluid flow director including a valve.

FIG. 11 is a cross section view of the fluid flow director with the valve of FIG. 11 in open position.

FIG. 12 is a cross section view of the open valve of FIG. 11 taken along lines A-A.

FIG. 13 is a cross section view of the valve of FIG. 10 in closed position.

FIG. 14 is a cross section side and top view of a fluid flow director including a septum having a duck bill oriented at 30 degrees.

FIG. 15 is a side, top, and end view of a fluid flow director including a septum having a duck bill oriented parallel to a fluid path.

FIG. 16 is a cross section view of a fluid flow director including a venturi.

FIG. 17 is a cross section view of a fluid flow director including a venturi.

FIG. 18 is a perspective cross section view of a fluid flow director including a pointed floor having an offset outlet hole.

FIG. 19 is a perspective partial cross section view of a fluid flow director including a ramp and a helical floor having an offset outlet hole.

FIG. 20 is a perspective cross section view of a fluid flow director including an offset outlet hole and a wall surrounding a portion of the offset outlet hole.

FIG. 21a is a cross section view of a fluid flow director including a turbine.

FIG. 21b is a top view of the turbine of FIG. 21a.

FIG. 21c is a graph showing variations in pressure over time.

FIG. 22 is a cross section view of a fluid flow director including an inlet torous and a goblet-shaped insert.

FIG. 23 is a cross section view of a fluid flow director including an outlet torous.

FIG. 24 is a cross section view of a fluid flow director including a cup-shaped barrier having an outlet at an edge of the cup-shaped barrier.

FIG. 25 includes multiple cross section side and top views of multiple fluid flow directors.

### DETAILED DESCRIPTION OF THE INVENTION

The presently preferred embodiments of the present invention will be best understood by reference to the drawings, wherein like reference numbers indicate identical or functionally similar elements. It will be readily understood that the components of the present invention, as generally described and illustrated in the figures herein, could be arranged and designed in a wide variety of different configurations. Thus, the following more detailed description, as represented in the figures, is not intended to limit the scope of the invention as claimed, but is merely representative of presently preferred embodiments of the invention.

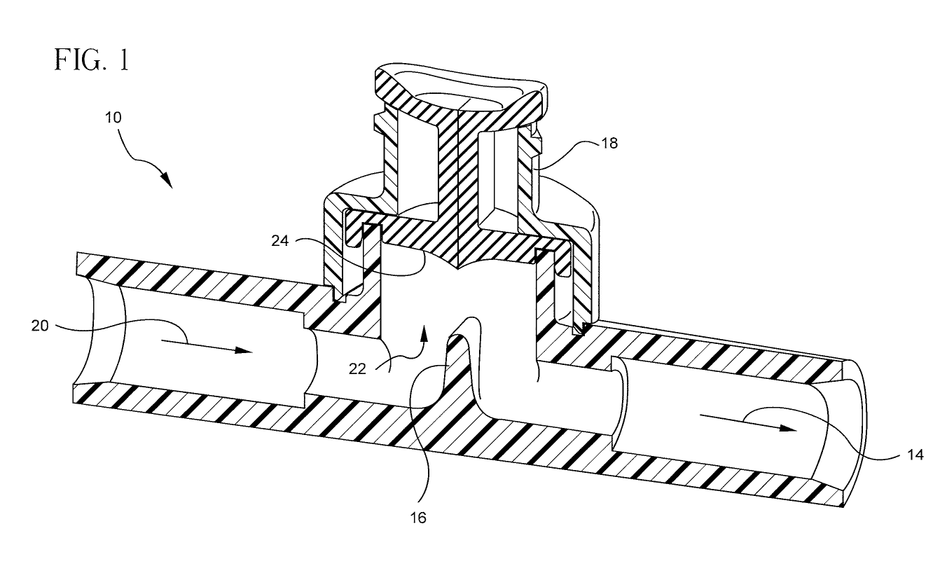

Referring now to FIG. 1, an extravascular system 10 includes a vascular access device 12 and is used to communicate a substance along a fluid path 14 of the system 10 with the vascular system of a patient. The extravascular system 10 includes a fluid flow director 16 in communication with the fluid path 14. The director 16 encourages the movement of stagnant fluid within the fluid path 14 of the system 10. The fluid flow director 16 is an arch located beneath the access port 18 of the vascular access device 12.

As fluid flows along the fluid path 14 in a direction 20, the fluid will come into contact with the arch-shaped director 16, causing the fluid to travel upwards in a direction 22 towards the bottom surface 24 of the access port 18. In the absence of the arched shaped fluid flow director 16, the fluid would continue to travel in the direction 20 past the bottom surface 24 of the access port 18, bypassing any stagnant fluid that may reside adjacent the bottom surface 24. The arch-shaped director 16, by contrast, forces the fluid in the direction 22 towards the stagnant fluid adjacent the bottom surface 24, encouraging the movement of stagnant fluid.

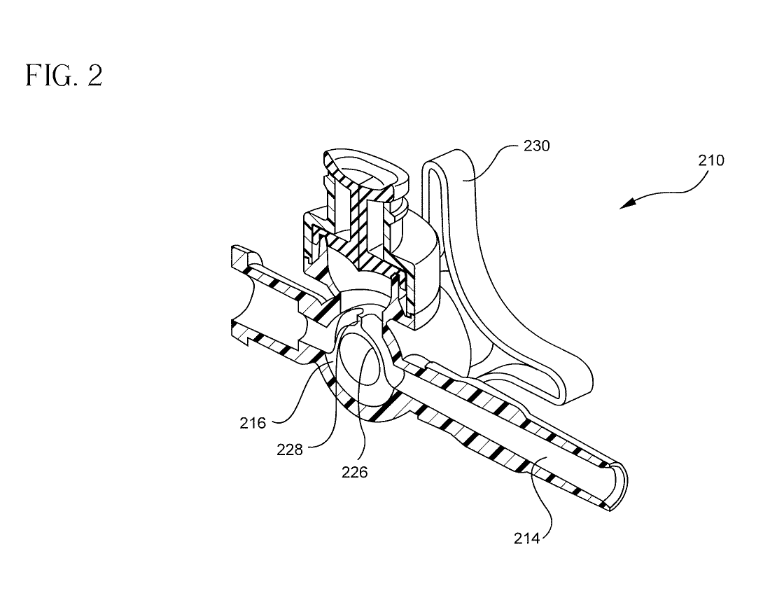

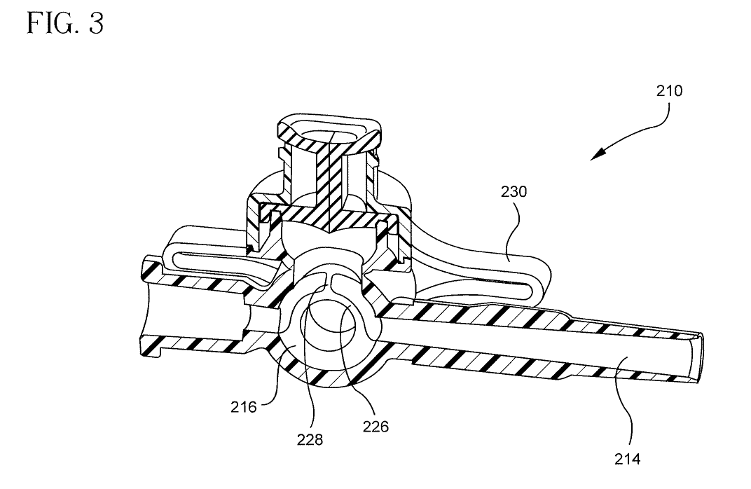

Referring now to FIG. 2, an extravascular system 210 includes a fluid path 214 and a fluid flow director 216 in communication with the fluid path 214. The director 216 encourages the movement of stagnant fluid within the fluid path 214 of the system 210. The director 216 includes a rotatable arch 226 with a lip 228 at the tip of the arch. The lip 228 forces fluid traveling along the outer surface of the arch 226 in a direction away from the arch 226. The lip 228 may thus be used to direct fluid towards an area of stagnant fluid within the fluid path 214 of the system 210. An operator may rotate a handle 230, which is affixed to the director 216, to rotate the director 216, enabling the operator to encourage the movement of stagnant fluid residing away from the arch 226 by steering the lip 228. Referring now to FIG. 3, the extravascular system 210 of FIG. 2 is shown in cross section perspective view.

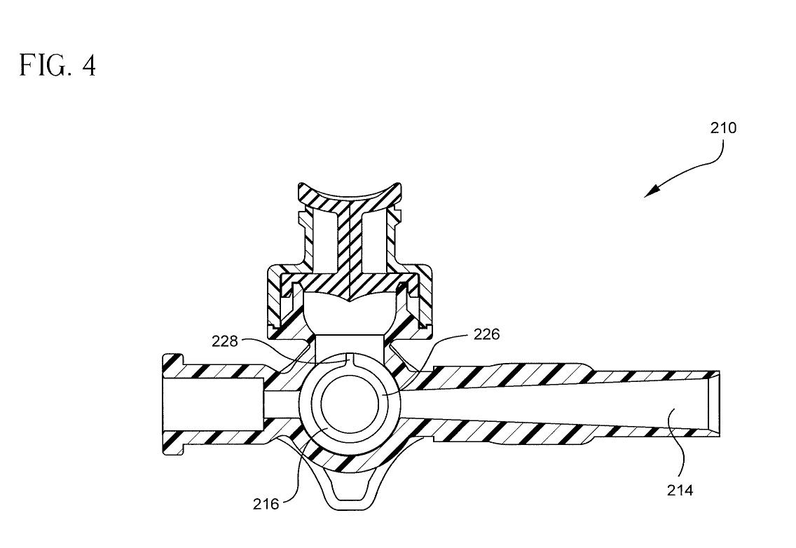

Referring now to FIG. 4, the extravascular system 210 of FIGS. 2 and 3 is shown in cross section view.

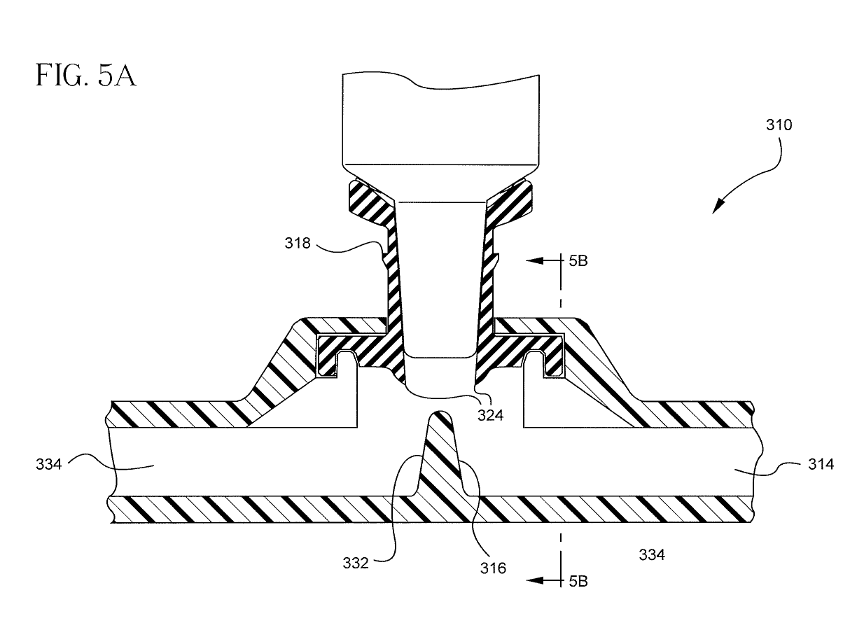

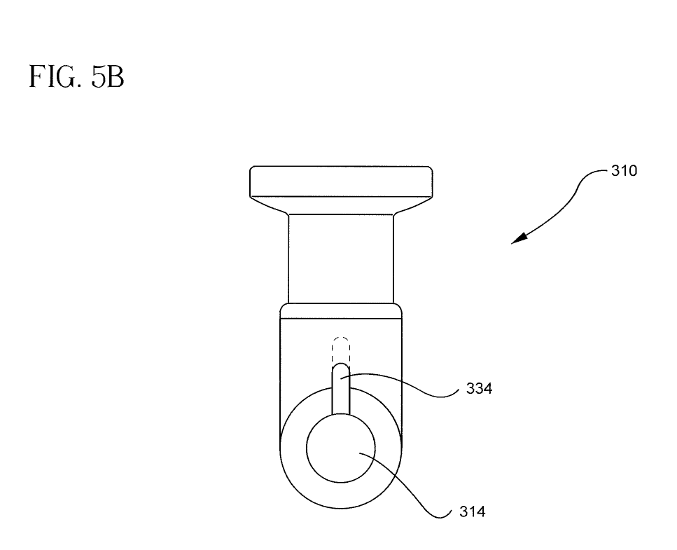

Referring now to FIG. 5, an extravascular system 310 includes a fluid path 314 and a fluid flow director 316 in communication with the fluid path 314. The fluid flow director 316 encourages the movement of stagnant fluid within the fluid path 314 of the system 310. The fluid flow director 316 includes an arch 332 and a flow channel 334 on either side of the arch 332. The arch 332 interrupts the straight path of the fluid path 314 and is located opposite the access port 318 of a vascular access device 312 that is secured to the extravascular system 310. The flow channels 334 are located opposite the arch 332 and adjacent the bottom surface 324 of the access port 318. The flow channels 334 direct fluid from the fluid path 314, through a first flow channel 334, towards a first bottom surface 324, over the arch 332, past a second surface 324, through a second flow channel 334, and into the straight fluid path 314 of the system 310. FIG. 5a is an end view of a portion of the system 310 illustrates that the flow channel 334 is an elevated channel within the fluid path 314.

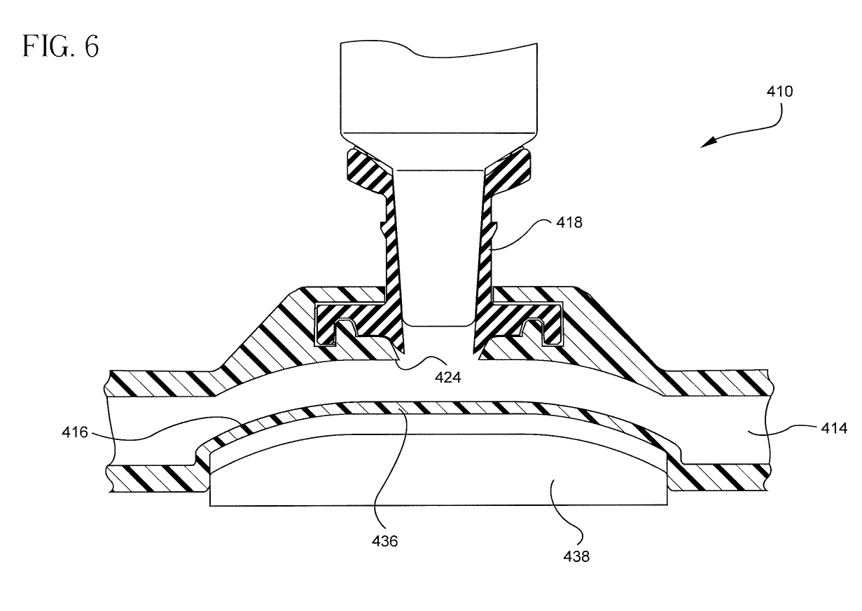

Referring now to FIG. 6, an extravascular system 410 includes a fluid path 414 and a fluid flow director 416 in communication with the fluid path 414. The director 416 encourages movement of stagnant fluid within the fluid path 414 of the system 410. The director 416 includes an arch 436 that defines a radial fluid path. The raised surface of the arch 436 causes fluid to travel from an otherwise straight fluid path 414 up and over the arch 436 towards the bottom surface 424 of an access port 418 secured to the system 410. Stagnant fluid that would otherwise reside adjacent the bottom surface 424 is moved as a result of fluid traveling through the altered fluid path 414.

The raised arch 436 may be solvent bonded or sonic welded to the system 410. A bottom cap 438 may be secured to the arch 436 in order to provide material that is easily molded in order to form the arch 436 during manufacture. Any portion of the access port 418 in communication with the fluid path 414 may be rotated or oriented in any direction in order to encourage the movement of stagnant fluid adjacent the bottom surface 424.

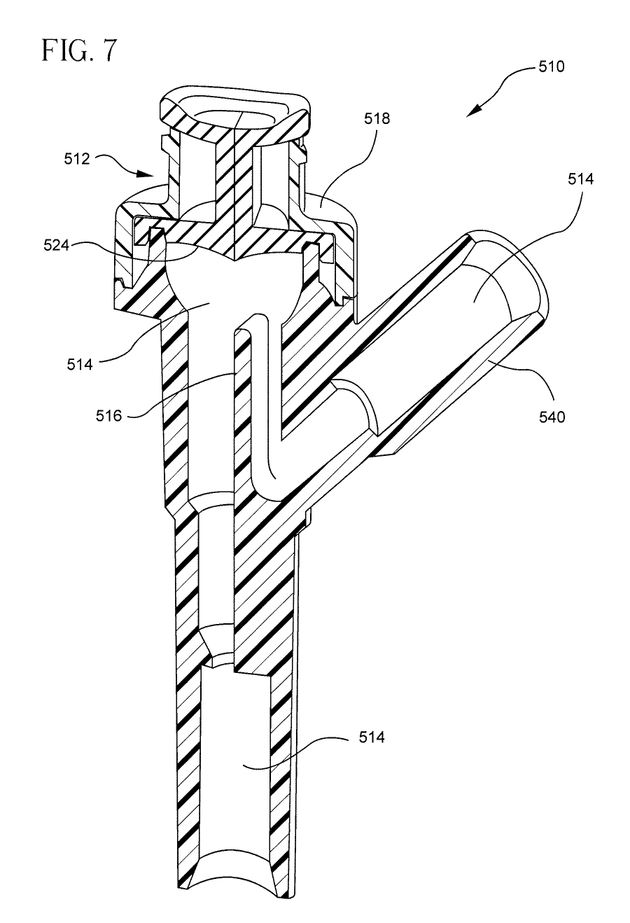

Referring now to FIG. 7, an extravascular system 510 includes a fluid path 514 and a fluid flow director 516 in communication with the fluid path 514. The director 516 encourages the movement of stagnant fluid within the fluid path 514 of the system 510. The fluid flow director 516 includes an arm extending between two portions of the fluid path 514 in a direction towards the access port 518 of a vascular access device 512 secured to the system 510. The arm of the fluid flow director 516 encourages fluid to flow through the fluid path 514 from a Y extension 540 secured to the system 510 towards any stagnant fluid that may reside adjacent the bottom surface 524 of the access port 518.

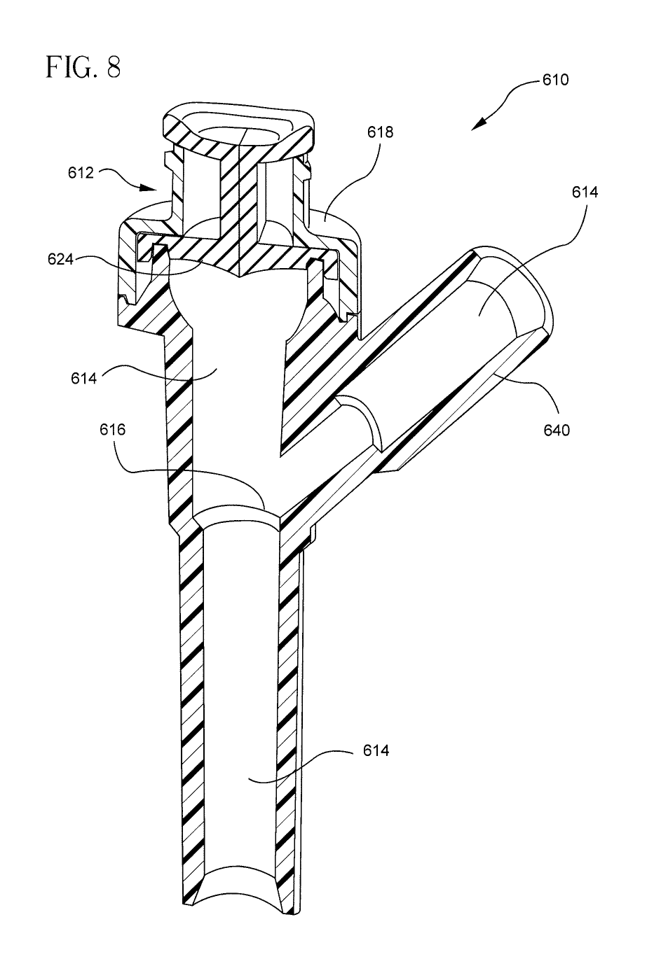

Referring now to FIG. 8, an extravascular system 610 includes a fluid path 614 and a fluid flow director 616 in communication with the fluid path 614. The director 616 encourages the movement of stagnant fluid within the fluid path 614 of the system 610 in an area adjacent the bottom surface 624 of an access port 618 secured to the system 610. The fluid flow director 616 includes an offset input.

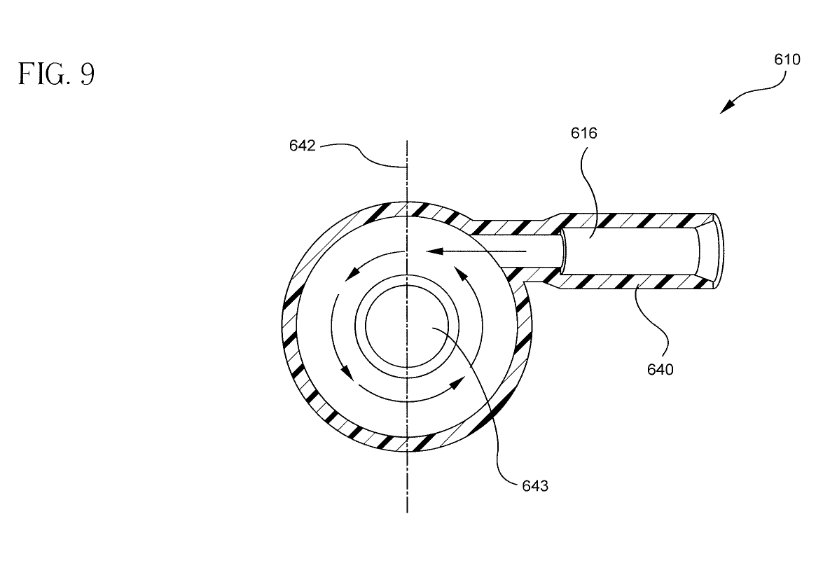

Referring now to FIG. 9, a cross section view taken along lines A-A of FIG. 8 is shown. The cross section view reveals the offset input of the fluid flow director 616 at a location off of a center line 642. The offset input extending from a Y extension 640 channels fluid along the fluid path 614 into a chamber 643 of the system 610, causing the fluid to travel in a circular motion around the inner surface of the system 610 within the chamber 643. As the fluid travels in a circular motion within the chamber 643, any fluid that would be stagnant absent the fluid flow director 616 is encouraged to move as a result of the circular motion of the fluid path 14.

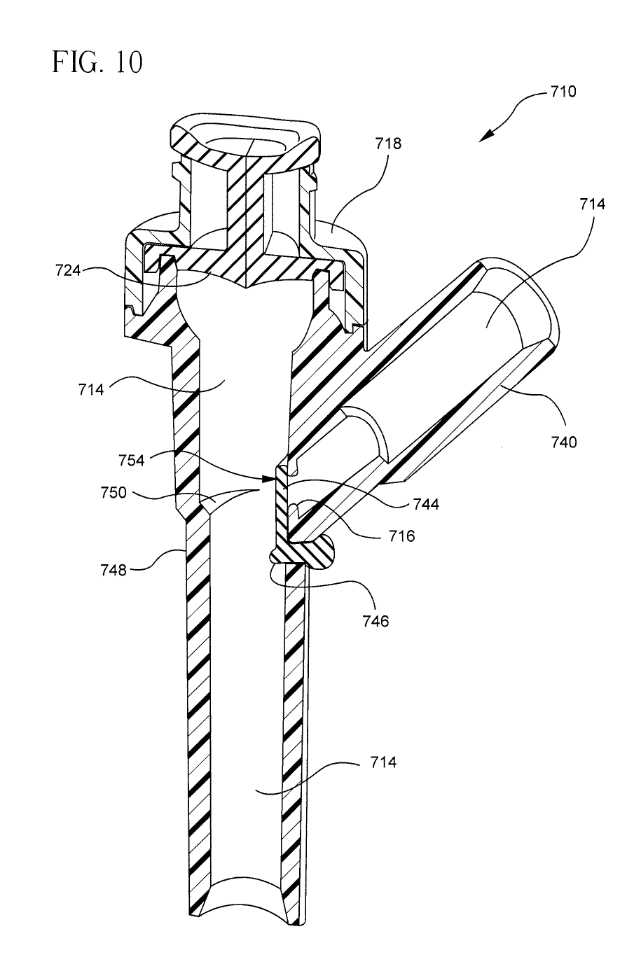

Referring now to FIG. 10, an extravascular system 710 includes a fluid path 714 and a fluid flow director 716 in communication with the fluid flow path 714. The director 716 encourages movement of stagnant fluid within the fluid path 714 of the system 710. The fluid flow director 716 includes a valve 744. The valve 744 may be formed of an elastomeric material in the shape of a disc capable of pivoting upon a point 746 at which the valve 744 is attached to the body 778 of the system 710.

The valve 744 is capable of moving from a first position in which the fluid path 714 of a Y extension 740 is closed or substantially closed to the remaining fluid paths 714 of the system 710. The valve 744 may move from the first position to a second position. In a second position, the valve 744 rests against a rib 750 secured to the body 748 of the system 710 at a location across the fluid path 714 and opposite the point 746.

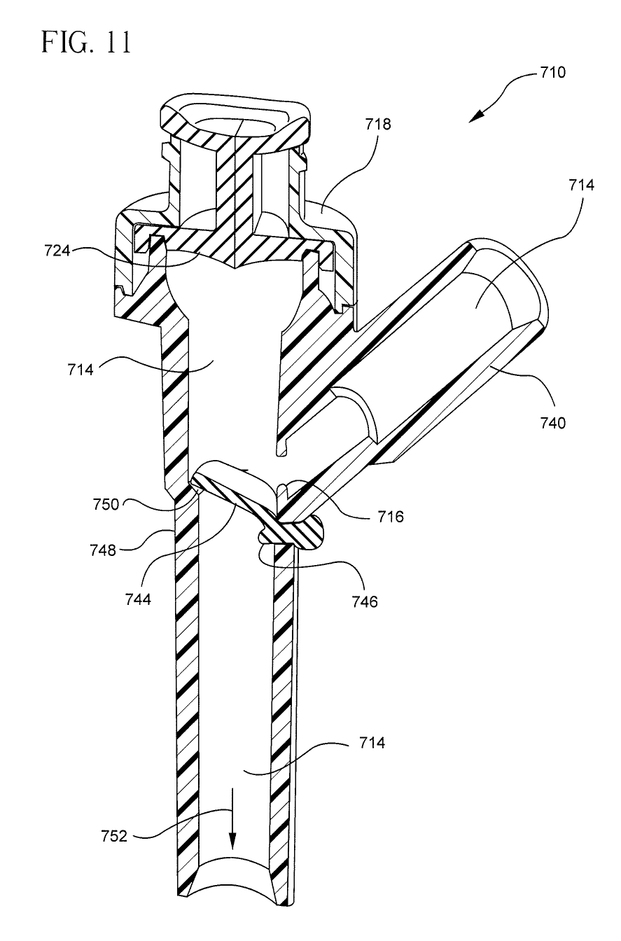

Referring now to FIG. 11, the extravascular system 710 of FIG. 10 is shown with the valve 744 in its second position, resting against the rib 750, and providing fluid communication between all portions of the fluid path 714. In its second position, the valve 744 of the fluid flow director 716 forces fluid to travel from the Y extension 740 towards the bottom surface 724 of an access port 718 secured to the system 710. The fluid is then forced between the valve 744 and the body 748, around the rib 750, in a direction 752 towards the vascular system of a patient.

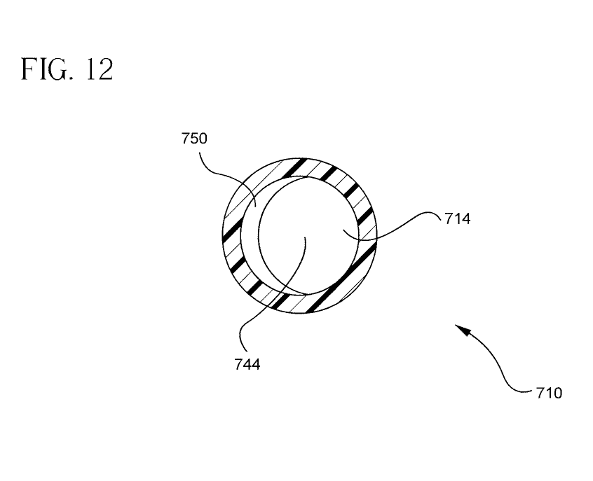

Referring now to FIG. 12, a cross section taken along lines A-A of the extravascular system 710 of FIG. 11 is shown. The valve 744 is closed, resting against the rib 750. Additional space between the valve 744 and the body 748, around the rib 750, permits the fluid path 714 to channel fluid through the system 710.

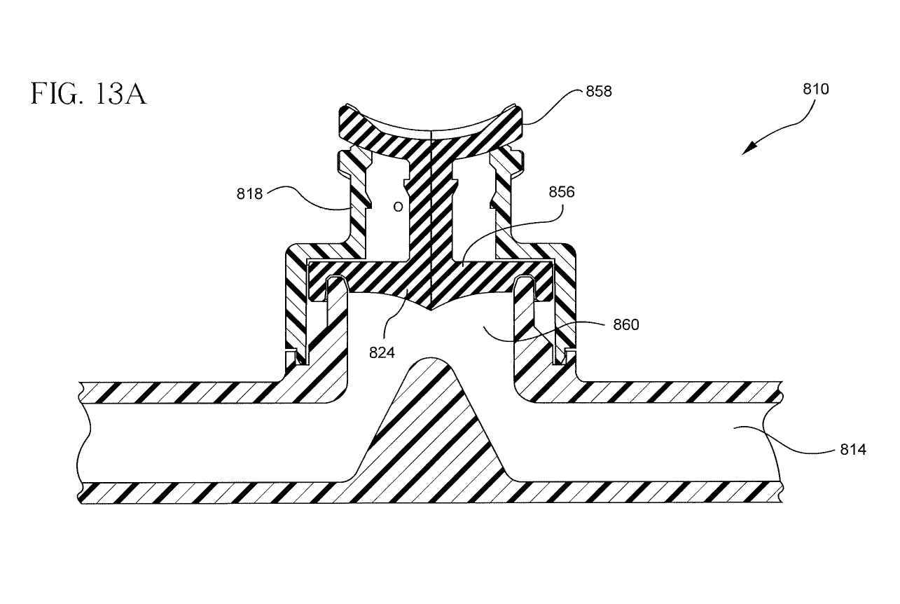

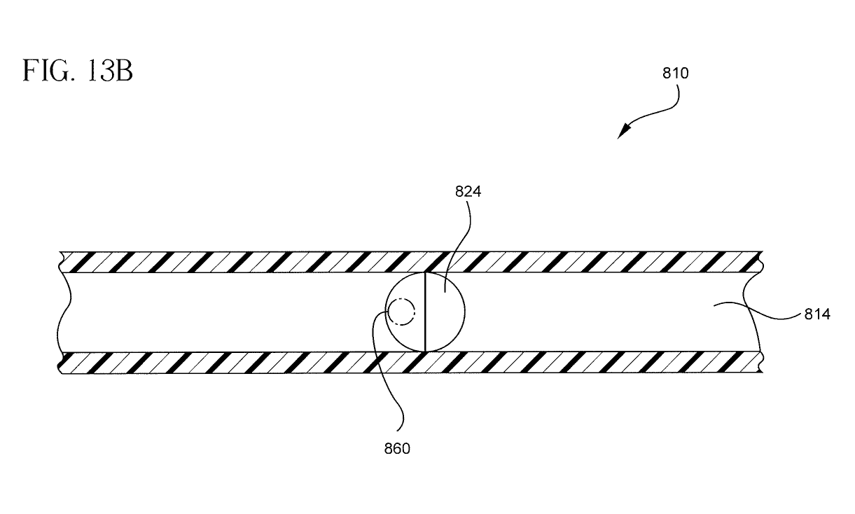

Referring now to FIG. 13, the extravascular system 710 of FIGS. 10 through 12 is shown with the valve 744 in its original first position after having been in its second position as shown in FIGS. 11 and 12. The valve 744 has returned to its original first position both as a result of the back pressure 754 from fluid within the fluid path 714 of the system 710 and as a result of the resiliency of the elastomeric material at the point 746. In its first position, the valve 744 prevents fluid from traveling from within a chamber 743 into the fluid path 714 of the Y extension 740.

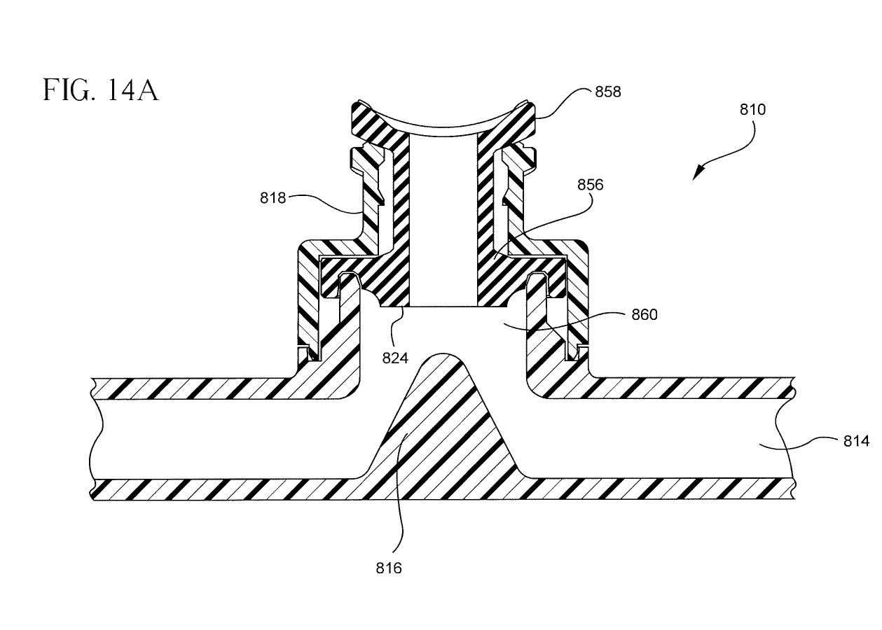

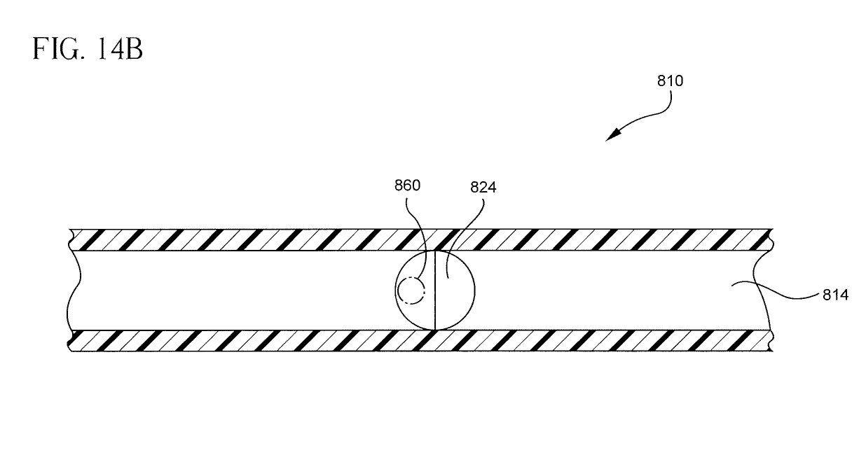

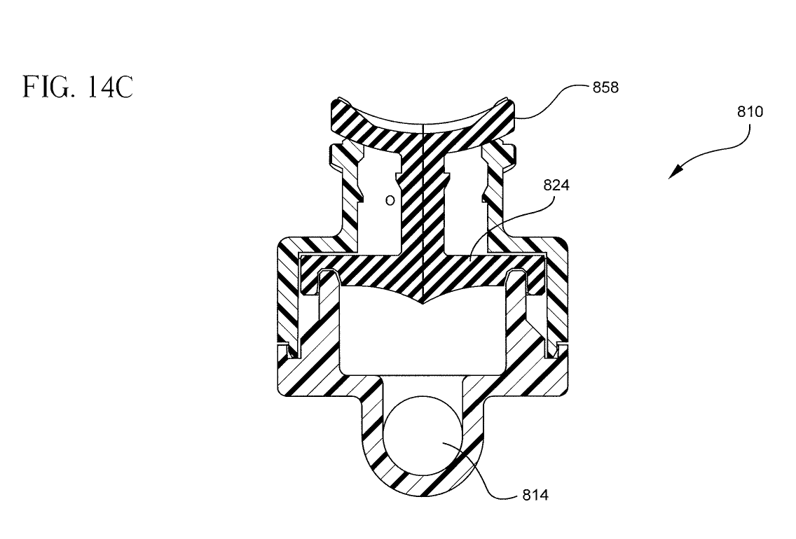

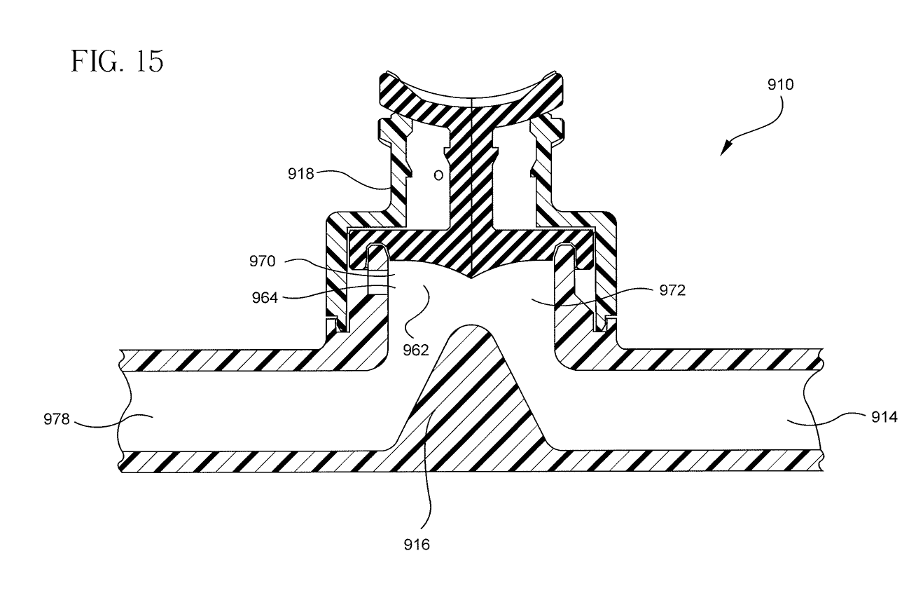

Referring now to FIG. 14, an extravascular system 810 includes a fluid path 814 in communication with the bottom surface 824 of the duck bill 856 of a septum 858 of a Luer access port 818. A top cross section view of the system 810 reveals that the duck bill 856 is oriented 30 degrees off from the axis of the fluid 814. In this particular orientation, stagnant fluid, such as air bubbles or residual medications may become trapped in an area of stagnant fluid 860 adjacent to the bottom surface 824 of the septum 858. As fluid flows through the fluid path 814, the particular orientation of the duck bill 856 is unlikely to permit stagnant fluid to be released from the area 860. Therefore, an embodiment reorienting the duck bill 856 of the septum 58 in order to release stagnant fluid from the area 860 may be preferred and will be described with reference to FIG. 15.

Referring now to FIG. 15, an extravascular system 810 includes a fluid path 814 in communication with the bottom surface 824 of a duck bill 856 of a septum 858. The duck bill 856 is oriented parallel with the flow of the fluid through the fluid path 814 as illustrated in a side view, top view, and end view of the system 810. With the duck bill 856 oriented parallel to the flow of fluid through the fluid path 814, no areas of stagnant fluid such as the area 860 illustrated in FIG. 14 are likely to develop, since the surface 824 of the duck bill 856 that protrudes into the fluid path 814 is not set at an angle capable of damming or otherwise blocking the natural flow of fluid through the fluid path 814.

The embodiment described with reference to FIG. 15 thus describes a fluid flow director 816 in communication with a fluid path 814. The director 816 encourages the movement of stagnant fluid within the fluid path 814 of the extravascular system 810. The fluid flow director 816 includes a septum 858 having a duck bill 856 that is oriented parallel to the fluid path 814.

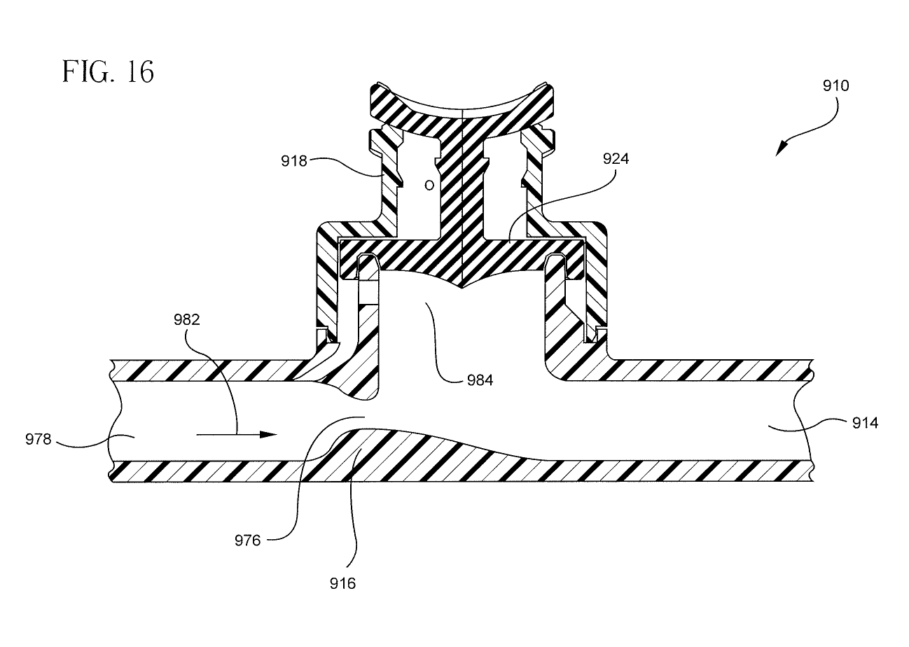

Referring now to FIG. 16, an extravascular system 910 includes a fluid path 914 and a fluid flow director 916 in communication with the fluid path 914. The director 916 encourages the movement of stagnant fluid 962 within the fluid path 914 of the extravascular system 10. The fluid flow director 916 includes a venturi.

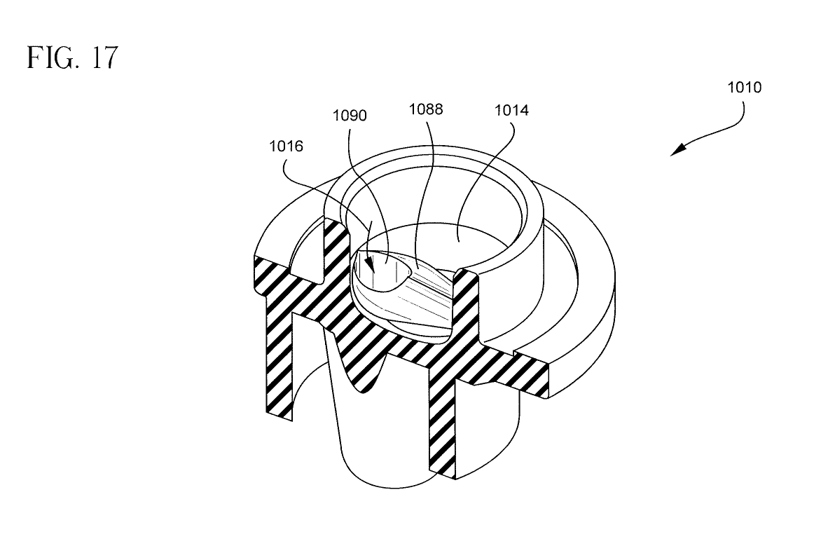

The venturi includes a major fluid flow path 964, a minor fluid flow path 966, an arch 968 within the major fluid flow path 964, an area of high pressure 970 within the fluid path 914, and an area of low pressure 972 within the fluid path 914. As fluid flows in a direction 974 through the fluid path 914, the majority of fluid will travel through the major fluid flow path 964 and the minority of fluid will flow through the minor fluid flow path 966. The major flow path 964 will rejoin the minor flow path 966 where the areas of high pressure 970 and low pressure 972 converge. The area of high pressure 970 will draw fluid from the area of low pressure 972 and consequently fluid from the minor 968 fluid flow path 966, causing fluid to flow in the area of stagnant fluid 962 back into the active fluid path 914. In this manner the fluid flow director 916 is capable of encouraging the movement of stagnant fluid within the fluid path 914. A similar embodiment is described with reference to FIG. 17.

Referring now to FIG. 17, an extravascular system 910 includes a fluid path 914 and a fluid flow director 916 in communication with the fluid path 914. The fluid flow director 916 encourages the movement of stagnant fluid within the fluid path 914 of the extravascular system 910. The fluid flow director 916 includes a venturi having a tapered diameter 976 in a major fluid flow path 978. The tapered diameter 976 is present in the fluid path 914 downstream of a minor fluid path 980 inlet. As fluid travels in a direction 982 within the fluid path 914, it will slow as it bottlenecks within the tapered diameter 976, causing pressure to build upstream the diameter 976 within the fluid path 914. As pressure builds within the fluid path 914, fluid will be forced into the inlet of the minor fluid path 980. Fluid will then travel at a relatively high speed through the minor fluid flow path 980 into an area of low pressure 984.

The area of low pressure 984 is an area within the fluid path 914 where stagnant fluid would tend to reside adjacent the bottom surface 924 of an access port 918. By receiving fluid through the minor fluid flow path 980 at a high speed, the area of lower pressure 984 will be flushed from any stagnant fluid residing therein. Further, an area of high pressure 986 within the major fluid flow path 978 will receive or otherwise draw fluid from the area of low pressure 84 back into the main fluid path 914.

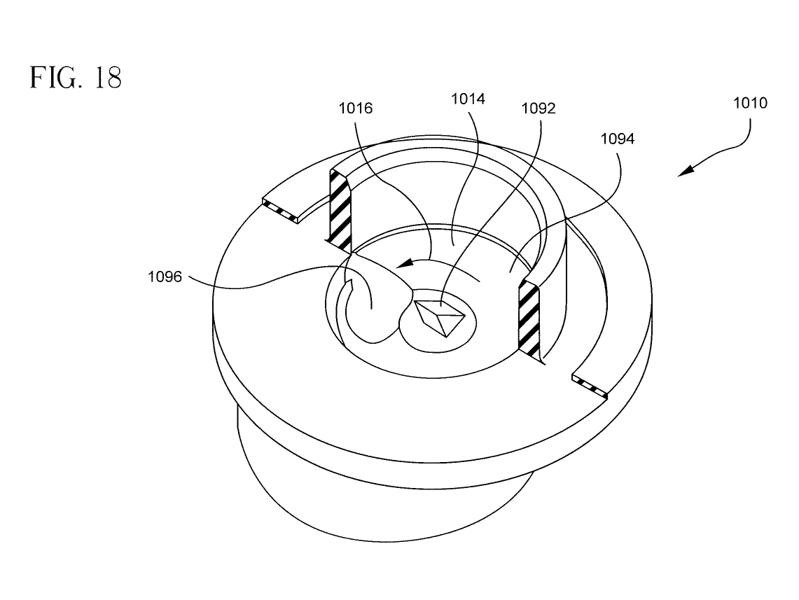

Referring now to FIG. 18, an extravascular system 1010 includes a fluid path 1014 and a fluid flow director 1016 in communication with the fluid path 1014. The fluid flow director 1016 encourages the movement of stagnant fluid within the fluid path 1014 of the extravascular system 1010. The fluid flow director 1016 includes a pointed and downward sloping floor 1088 adjacent an offset fluid outlet hole 1090.

As fluid travels through the center of the system 1010 along the fluid path 1014 towards the fluid flow director 1016, a majority of the fluid will come into contact with the pointed and downward sloping floor 1088. As fluid comes into contact with the floor 1088, the fluid will separate and spread throughout a chamber within the system 1010. Various currents within the chamber of the system 1010 will circulate until the fluid is able to find its escape through the offset fluid outlet hole 1090. During the circulation of current throughout the whole volume of a chamber within the system 1010, any fluid that would otherwise be stagnant will be encouraged to move and enter into the active fluid path 1014, ultimately being flushed through the offset outlet hole 1090. Various embodiments alternate to the embodiment described with reference to FIG. 18 are possible and will be described with reference to the following figures.

Referring now to FIG. 19, an extravascular system 1010 includes a fluid path 1014 and a fluid flow director 1016 in communication with the fluid path 1014. The director 1016 encourages movement of stagnant fluid within the fluid path 1014 of the extravascular system 1010. The fluid flow director 1016 includes a ramp 1092 and a helical floor 1094 having an offset outlet hole 1096.

As fluid travels through the center of the fluid path 1014 of the system 1010 towards the fluid flow director 1016, a majority of the fluid in the fluid path 1014 will come into contact with the ramp 1092. The ramp 1092 will then direct the fluid from the fluid path 1014 towards an upper portion of the tapered helical floor 1094. The tapered helical floor 1094 will then direct the fluid in a helical motion around the volume of a chamber within the system 1010 ultimately towards the offset outlet hole 1096 at the end of the helix of the helical floor 1094. The offset outlet hole 1096 will then receive the fluid from the fluid path 1014 and direct it further within the system 1010. In this manner, the fluid flow director 1016 is capable of circulating fluid within an entire volume of a chamber of the system 1010 in a manner that encourages movement of any stagnant fluid contained therein.

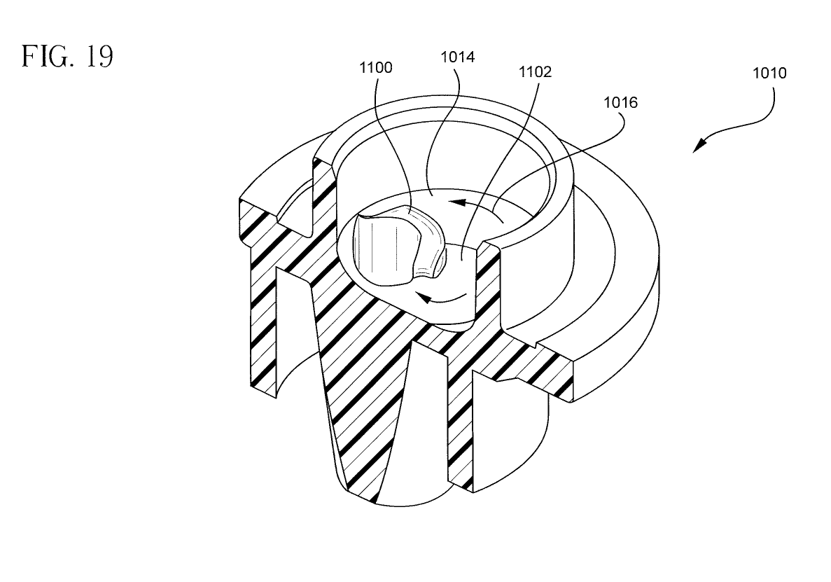

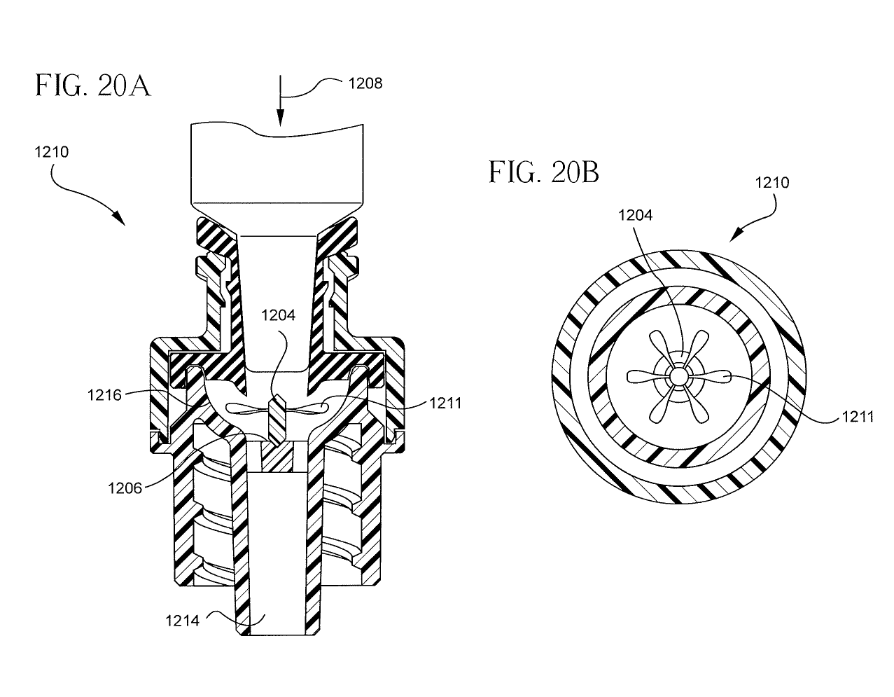

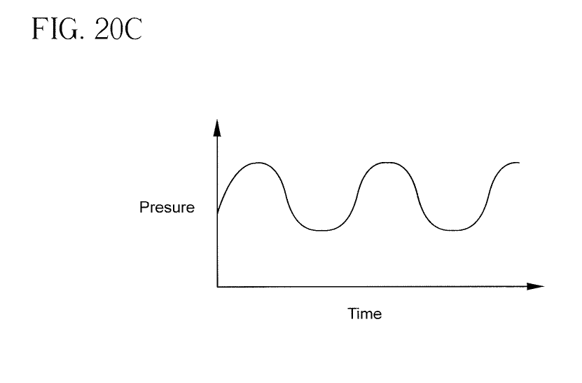

Referring now to FIG. 20, an extravascular system 1010 includes a fluid path 1014 and a fluid flow director 1016 in communication with the fluid path 1014. The fluid flow director 1016 encourages movement of stagnant fluid within the fluid path 1014 of the system 1010. The fluid flow director includes an offset hole 1098 and a wall 1100 surrounding a portion of the offset hole 1098.

As fluid travels through the center of the system 1100 along fluid path 1014, fluid will enter into the chamber containing the fluid flow director 1016. As the fluid travels through the chamber towards the fluid flow director 1016, a majority of the fluid will come into contact with a floor 102 of the chamber. The wall 1100 separates the portion of the floor 1102 that first receives the majority of the fluid from the offset outlet hole 1098. Thus fluid being forced against the floor 1102 will be forced to travel along the floor 1102, around the wall 1100, and into the offset hole 1098 in order to travel through the remaining fluid path 1014 of the system 1010. As fluid travels in its circuitous path along the floor 1102 towards the offset outlet hole 1098, any stagnant fluid that would otherwise reside within the chamber containing the fluid flow director 1016 will be encouraged to move and ultimately enter into the offset outlet hole 1098.

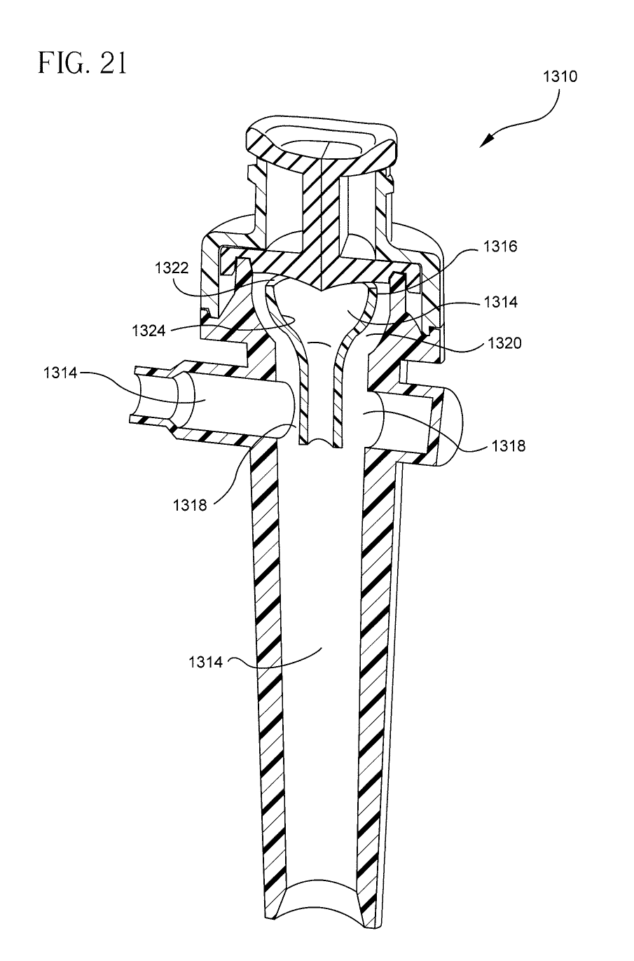

Referring now to FIGS. 21a-c, an extravascular system 1210 includes a fluid path 1214 and a fluid flow director 1216 in communication with the fluid path 1214. The fluid flow director 1216 encourages the movement of stagnant fluid within the fluid path 1214 of the system 1210. The fluid flow director 1216 includes a turbine 1204. The turbine 1204 is a semi-floating turbine resting on a cone-in-cone bearing 1206 within a chamber of the system 1210.

As fluid travels through the fluid path 1214 in a direction 1208, the fluid will force the turbine 1204 to spin within the chamber containing the fluid flow director 1216. As the turbine 1204 spins, the wings 1211 of the turbine will generate turbulence within the chamber. The turbulence will cause fluid that would otherwise be stagnant to move and enter into the active flow of the fluid path 1214. Further, as the wings 1211 of the turbine 1204 circulate past areas of likely stagnant fluid 1212, each passing wing 1211 will create an increase of pressure followed by a decrease in pressure. The wings 1211 will thus provide a recurring, pulsating pressure profile due to the passing wings 1211.

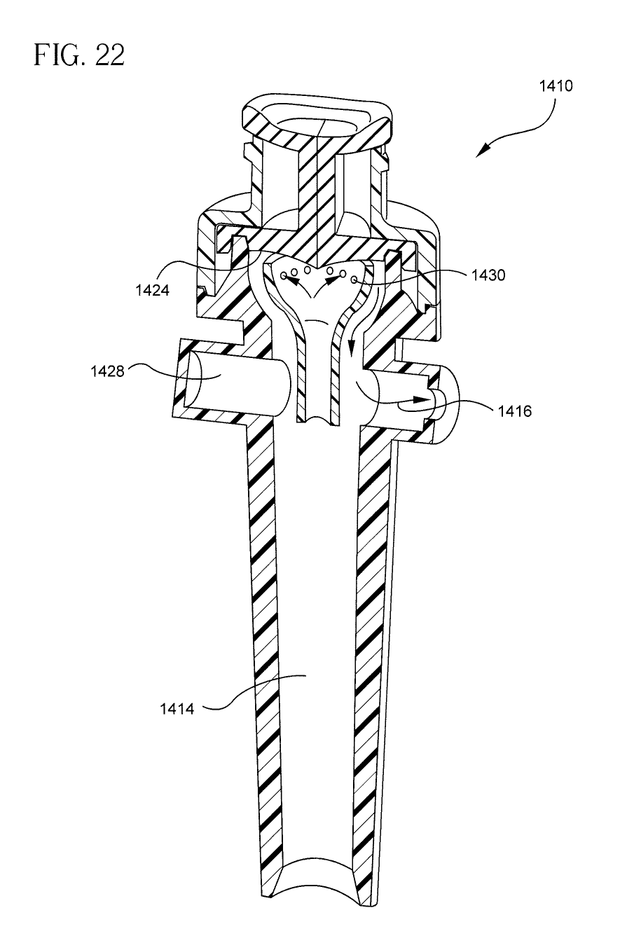

Referring now to FIG. 22, an extravascular system 1310 includes a fluid path 1314 and a fluid flow director 1316 in communication with the fluid path 1314. The director 1316 encourages the movement of stagnant fluid within the fluid path 1314 of the extravascular system 1310. The fluid flow director 1316 includes an inlet torous 1314 and a goblet-shaped insert 1316.

The fluid inlet torous 1314 receives fluid through the fluid path 1314, directs fluid through fluid inlet holes 1318 around the body of the system 1310, into a fluid path on an outer surface 1320 of the goblet-shaped insert 1316, above and around a top lip 1322 of the goblet-shaped insert 1316, past an inner surface 1324 of the goblet-shaped insert 1316, and ultimately through the remaining fluid path 1314 of a fluid outlet 1326. By providing a circuitous path through which fluid must flow in the fluid path 1314, the fluid flow director 1316 ensures that stagnant fluid is encouraged to move from the inlet torous 1314 to the fluid outlet 1326. Further, since the goblet-shaped insert 1316 directs fluid to the lip 1322, and since the lip 1322 adjacent the bottom surface 1324 of an access port 1318, any stagnant fluid that would traditionally reside beneath the bottom surface 1324 will be forced to move into the active fluid path 1314.

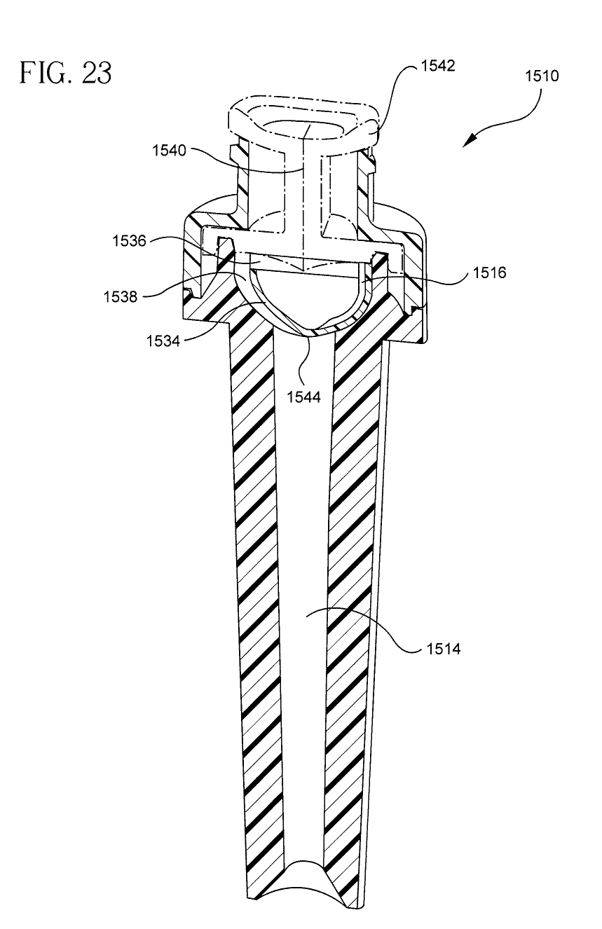

Referring now to FIG. 23, an extravascular system 1410 includes a fluid path 1414 and a fluid flow director 1416 in communication with the fluid path 1414. The fluid flow director 1416 encourages the movement of stagnant fluid within the fluid path 1414 of the system 1410. The fluid flow director 1416 includes an outlet torous 1428.

The outlet torous 1428 is located in the system 1410 adjacent a bottom surface 1424 of an access port 1418. Fluid may enter the outlet torous 1428 through multiple holes 1430 connecting a chamber of the system 1410 with the outlet torous 1428. The outlet torous in turn is connected to a fluid outlet 1432 in a manner that permits fluid to flow through the fluid path 1414 from the outlet torous 1428 into the fluid outlet 1432. The holes 1430 are spaced throughout the chamber adjacent the bottom surface 1424 so as to ensure the flow of fluid through the holes 1430 in an area where stagnant fluid would otherwise reside if the holes 1430 were not present. Thus, the embodiment described with reference to FIG. 23 provides a fluid flow director 1416 capable of encouraging the movement of stagnant fluid along a fluid path 1414, through holes 1430, past a bottom surface 1424, into an outlet torous 1428, and into a fluid outlet 1432.

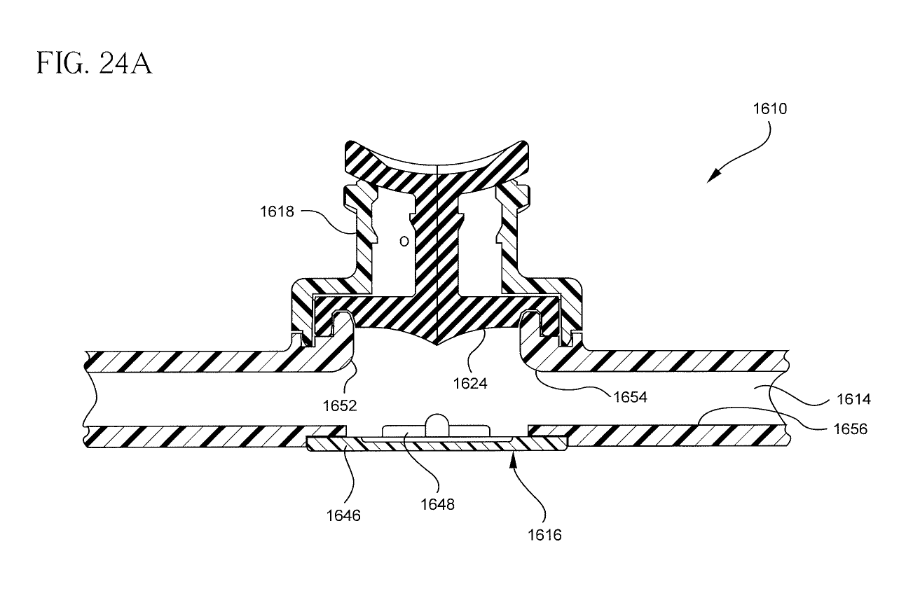

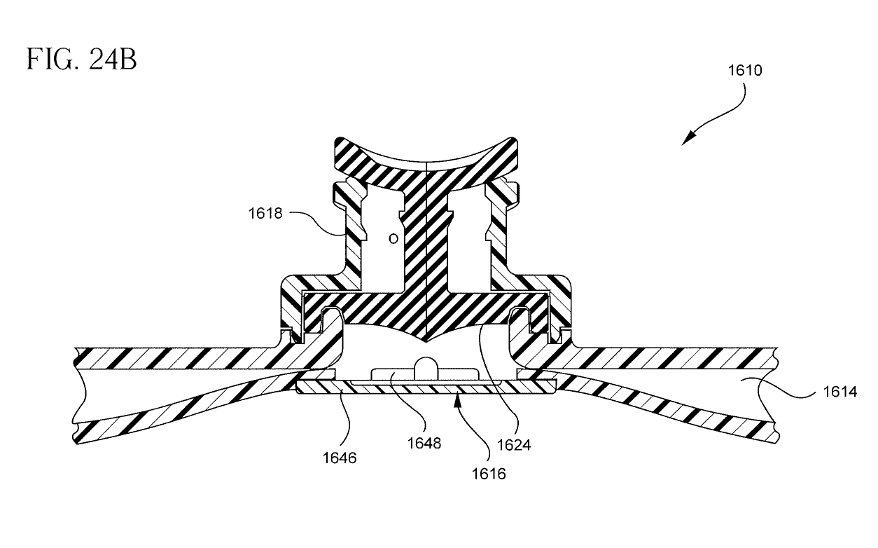

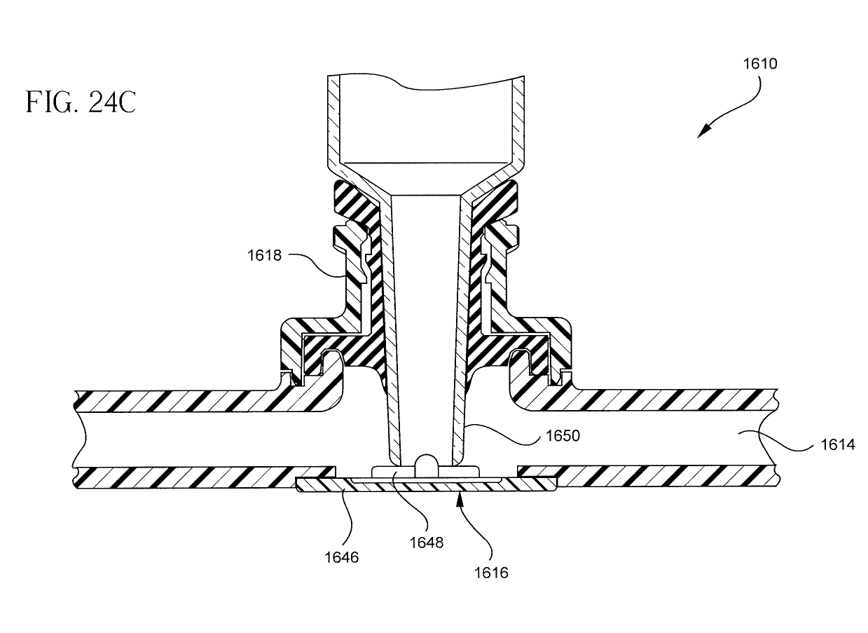

Referring now to FIG. 24, an extravascular system 1510 includes a fluid path 1514 in communication with a fluid flow director 1516. The fluid flow director 1516 encourages the movement of stagnant fluid within the fluid path 1514 of the system 1510. The fluid flow director 1516 includes a cup-shaped barrier 1534 having an outlet 1536 at the edge of the cup 1534. The cup-shaped barrier 1534 also includes a channel 1538 along the inner edge of the cup-shaped barrier 1534 that is opposite the outlet 1536.

In use, fluid will be infused through the slit 1540 of a septum 1542 into the chamber of the cup-shaped barrier 1534. The fluid will travel towards the bottom 1544 of the cup-shaped barrier 1534 and will turn to make its way up the channel 1538 towards the top end of the cup-shaped barrier 1534 that is opposite the outlet 1536. The fluid will then travel from the opposite end of the cup-shaped barrier 1534 towards the outlet 1536. Upon reaching the outlet 1536, fluid will travel outside the cup-shaped barrier 1534 and downward towards the remaining fluid path 1514 of the extravascular system 1510. The fluid flow director 1516 described with reference to FIG. 24 thus provides a director 1516 capable of encouraging fluid movement throughout an entire chamber within a cup-shaped barrier 1534.

Referring now to FIG. 25, an extravascular system 1610 includes a fluid path 1614 and a fluid flow director 1616 in communication with the fluid path 1614. The fluid flow director 16 encourages the movement of stagnant fluid within the fluid path 1614 of the extravascular system 1610. The fluid flow director 16 includes a deflectable membrane 1646 and a Luer tip receiver 1648.

The deflectable membrane 1646 resides in communication with the fluid path 1614 opposite the bottom surface 1624 of an access port 1618. Upon access of a vascular access device through the access port 1618, the deflectable membrane 1646 may expand or stretch as a result of the tip of the vascular access device exerting pressure against the Luer tip receiver 1648. The Luer tip receiver 1648 is capable of receiving the tip of a Luer 1650.

In order to prevent the surface of the tip 1650 from forming a seal against the Luer tip receiver 1648, the Luer tip receiver 1648 is formed having gaps between certain portions of its structure. Those gaps are an absence of material that a Luer tip 1650 would normally seal against in the presence of such material. However, since the Luer tip receiver 1648 includes at least one gap in its material, fluid may flow from the Luer tip 1650 past the Luer tip receiver 1648 into the fluid path 1614. The gaps may be strategically positioned in order to minimize any stagnant fluid that may reside near the bottom surface 1624 of the access port 1618.

Any of the embodiments described with reference to any of the figures above, may incorporate any of the elements or features of any of those embodiments in combination and in any number in order to achieve the purposes of the present invention. Further, any embodiment may include any of the following surfaces and/or materials as a part of a fluid flow director 16: a hydrophobic surface to direct fluid away from a specific surface in a certain direction, a hydrophilic surface to attract fluid towards a surface and a specific direction, and/or a soluble or wicking material in order to attract fluid to a particular surface and in a certain direction. The soluble or wicking internal surface may include salt, sugar, cotton, or any other soluble or wicking material or substance. For illustration purposes, FIG. 25 includes at least one hydrophobic surface 1652, a hydrophilic surface 1654, and a wicking or soluble material 1656 on various inner surfaces in communication with the fluid path 1614. The hydrophobic, hydrophilic, and soluble surfaces 1652, 1654, and 1656, form part of a fluid flow director 1616.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

1. A medical device, comprising:
an extravascular system for communication of fluid with a vascular system;
a fluid path within the extravascular system; and
a fluid flow director in communication with the fluid path, wherein the director encourages the movement of stagnant fluid within the fluid path of the extravascular system.

2. The medical device of claim 1, wherein the fluid flow director includes an arch.

3. The medical device of claim 1, wherein the fluid flow director includes a rotatable arch with a lip at the tip of the arch.

4. The medical device of claim 1, wherein the fluid flow director includes an arch and a flow channel.

5. The medical device of claim 1, wherein the fluid flow director includes an arch that defines a radial fluid path.

6. The medical device of claim 1, wherein the fluid flow director includes an arm.

7. The medical device of claim 1, wherein the fluid flow director includes an offset input.

8. The medical device of claim 1, wherein the fluid flow director includes a valve.

9. The medical device of claim 1, wherein the fluid flow director includes a septum having a duck bill oriented parallel to the fluid path.

10. The medical device of claim 1, wherein the fluid flow director includes a venturi.

11. The medical device of claim 1, wherein the fluid flow director includes a pointed floor having an offset outlet hole.

12. The medical device of claim 1, wherein the fluid flow director includes a ramp and a helical floor having an offset outlet hole.

13. The medical device of claim 1, wherein the fluid flow director includes an offset outlet hole and a wall surrounding a portion of the offset outlet hole.

14. The medical device of claim 1, wherein the fluid flow director includes a turbine.

15. The medical device of claim 1, wherein the fluid flow director includes an inlet torus and a goblet shaped insert.

16. The medical device of claim 1, wherein the fluid flow director includes an outlet torus.

17. The medical device of claim 1, wherein the fluid flow director includes a cup-shaped barrier having an outlet at an edge of the cup-shaped barrier.

18. The medical device of claim 1, wherein the fluid flow director includes a deflectable membrane and a Luer tip receiver.

19. The medical device of claim 1, wherein the fluid flow director includes a hydrophilic material.

20. The medical device of claim 1, wherein the fluid flow director includes soluble material.

21. A method, comprising:
providing an extravascular system having a fluid path; and
encouraging the movement of stagnant fluid within the fluid path of the extravascular system by means of a fluid flow director.

22. A medical device, comprising:
a means for accessing the vascular system of a patient; and
a director means for encouraging the movement of stagnant fluid, wherein the director means for encouraging at least partially resides within the means for accessing the vascular system of the patient.
